(19) United States
(12) Patent Application Publication
(10) Pub. No.: US 2026/0263055 A1
(43) Pub. Date: Sep. 10, 2026

(54) URINE COLLECTION AND ANALYSIS ASSEMBLY

(71) Applicants: ARIZONA BOARD OF REGENTS ON BEHALF OF ARIZONA STATE UNIVERSITY, Scottsdale, AZ (US); MultiLucent Corp., Mesa, AZ (US)

(72) Inventors: David Wallace, Mesa, AZ (US); Firas Abbas, Mesa, AZ (US); Fredrick Zlaket, Scottsdale, AZ (US); Michelle DiPalma, Mesa, AZ (US); Ryan Stenborg, Mesa, AZ (US); Xavier Wallace, Mesa, AZ (US); Sydney Wallace, Mesa, AZ (US)

(21) Appl. No.: 19/556,836

(22) Filed: Mar. 4, 2026

Related U.S. Application Data
(60) Provisional application No. 63/767,903, filed on Mar. 6, 2025.

(51) Int. Cl. A61B 10/00; C12Q 1/18

(52) U.S. Cl. CPC A61B 10/007; C12Q 1/18; A61B 2010/009

(57) ABSTRACT

A urine collection and analysis assembly including: a specimen cup having a first collection chamber and a second collection chamber; and an assay base couplable with the specimen cup, the assay base including a first testing area including one or more testing substrates and a second testing area having one or more testing members, wherein coupling the specimen cup and the assay base fluidly connects the first testing area to the first collection chamber such that an initial void of urine runs over the one or more testing substrates for analyzing the initial void of urine and the second testing area to the second collection chamber such that a mid-stream-clean-catch of urine is exposed to the one or more testing members for analyzing the mid-stream-clean-catch.

### CROSS-REFERENCE TO RELATED APPLICATIONS

The present application claims priority to U.S. Provisional Patent Application No. 63/767,903, filed Mar. 6, 2025, the entire contents of which are incorporated herein by reference.

### FIELD

The present disclosure relates to urine sample collection containers, and more specifically to a urine sample collection container and testing assembly.

### BACKGROUND

Typical urine collection sample processes are both physically and mentally complex operations, which ultimately result in several opportunities for erroneous sample collection. For example, the Mid-Stream Clean Catch (MSCC) urine sample and collection technique requires a user to urinate into the toilet for a few seconds to discharge the initial void (IV) and then subsequently collect the remaining urine (the MSCC) until a certain volume is filled. It is often difficult for a user to determine how much volume they have discharged at any given time or to even maneuver the specimen cup in place to collect the urine while on the toilet. As a result, it is estimated that 70% of healthy women provide a contaminated sample. Furthermore, the typical process of collecting and analyzing urine samples requires several logistical steps. First, a user will drive to a doctor's office, where the doctor may either collect the urine sample and send the sample to be analyzed in a lab, or the doctor may instruct the user to travel to the lab itself to provide a urine sample.

### SUMMARY

In some aspects, the techniques described herein relate to a urine collection and analysis assembly including: a specimen cup having a first collection chamber and a second collection chamber; and an assay base couplable with the specimen cup, the assay base including a first testing area including one or more testing substrates and a second testing area having one or more testing members, wherein coupling the specimen cup and the assay base fluidly connects the first testing area to the first collection chamber such that an initial void of urine runs over the one or more testing substrates for analyzing the initial void of urine and the second testing area to the second collection chamber such that a mid-stream-clean-catch of urine is exposed to the one or more testing members for analyzing the mid-stream-clean-catch.

In some aspects, the techniques described herein relate to a specimen cup configured to receive a urine sample, the specimen cup including: an outer housing including a base wall; an outer wall coupled to and extending from the base wall, the outer wall defining a perimeter of the specimen cup; an inner housing positioned within the outer housing and including a first end; a second end opposite the first end and supported by the base wall of the outer housing; a valve element movably positioned with the inner housing; a first collection chamber defined by the inner housing and configured to receive an initial void of the urine sample; a second collection chamber defined within the outer housing between the inner housing and the outer wall, the second collection chamber in fluid communication with the first collection chamber and configured to receive a mid-stream-clean-catch of the urine sample; a first port positioned at the second end of the inner housing accessible through the base wall and configured to selectively open the first collection chamber; and a second port accessible through the base wall and configured to selectively open the second collection chamber.

In some aspects, the techniques described herein relate to an assay base couplable with a specimen cup configured to receive a urine sample, the assay base including: a housing having a base wall and a top wall; a first port actuator accessible through the top wall; a first testing area including one or more testing substrates; a first channel within the housing and in fluid communication between the first port actuator and the first testing area; a second port actuator accessible through the top wall; a second testing area having one or more testing members; and a second channel within the housing and in fluid communication between the second port actuator and the second testing area, wherein the first testing area is configured to receive an initial void of the urine sample, which runs over the one or more testing substrates for analyzing the initial void, and wherein the second testing area is configured to receive a mid-stream-clean-catch of the urine sample, which is selectively exposed to the one or more testing members for analyzing the mid-stream-clean-catch.

Other features and aspects will become apparent by consideration of the detailed description and accompanying drawings.

### BRIEF DESCRIPTION OF THE DRAWINGS

FIG. 1 illustrates a front perspective view of a urine collection and analysis assembly according to one embodiment including a specimen cup and an assay base.

FIG. 2 illustrates a perspective view of the specimen cup of FIG. 1.

FIG. 3 illustrates an exploded view of the specimen cup of FIG. 1.

FIG. 4 illustrates a perspective view of an inner housing with a first collection chamber of the specimen cup of FIG. 1.

FIG. 5 illustrates a perspective view of an outer housing of the specimen cup of FIG. 1.

FIG. 6 illustrates a perspective view of a saddle of the specimen cup of FIG. 1.

FIG. 7 illustrates a partially exploded view of the urine collection and analysis assembly of FIG. 1.

FIG. 8 illustrates a perspective view of the assay base of FIG. 1.

FIG. 9 illustrates an exploded view of the assay base of FIG. 1 with components.

FIG. 10 illustrates a bottom perspective view of the platform of the assay base of FIG. 1.

FIG. 11 illustrates a perspective view of the actuator of the assay base of FIG. 1.

FIG. 12 illustrates an exemplary side view of the platform, actuator, and testing members of the assay base of FIG. 1.

FIG. 13 illustrates a section view of the assay base according to another embodiment.

FIG. 14 illustrates a perspective view of a specimen cup and assay base assembly according to another embodiment.

FIG. 15 illustrates an exemplary region of exclusion resulting from antibiotics on a growth medium.

FIG. 16 illustrates a perspective view of a specimen cup according to another embodiment.

FIG. 17 illustrates a top view of the specimen cup of FIG. 16.

FIG. 18 illustrates a bottom view of the specimen cup of FIG. 16.

FIG. 19 illustrates a side view of the specimen cup of FIG. 16.

FIG. 20 illustrates a cross-sectional view of the specimen cup of FIG. 16.

FIG. 21 illustrates a top view of an assay base of FIG. 1.

FIG. 22 illustrates a cross-section view of the assay base of FIG. 21.

FIG. 23 illustrates a cross-sectional view of the assay base of FIG. 21

Before any embodiments are explained in detail, it is to be understood that the invention is not limited in its application to the details of construction and the arrangement of components set forth in the following description or illustrated in the accompanying drawings. The invention is capable of other embodiments and of being practiced or of being carried out in various ways. Also, it is to be understood that the phraseology and terminology used herein is for the purpose of description and should not be regarded as limiting.

### DETAILED DESCRIPTION

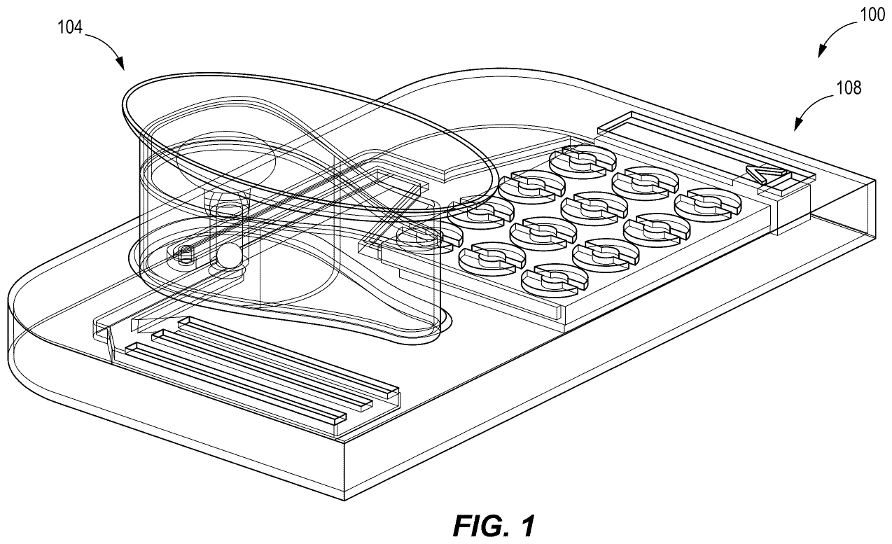

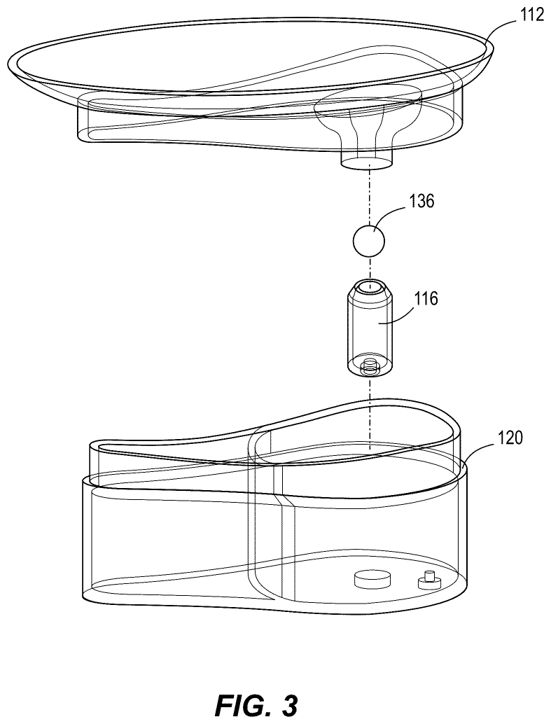

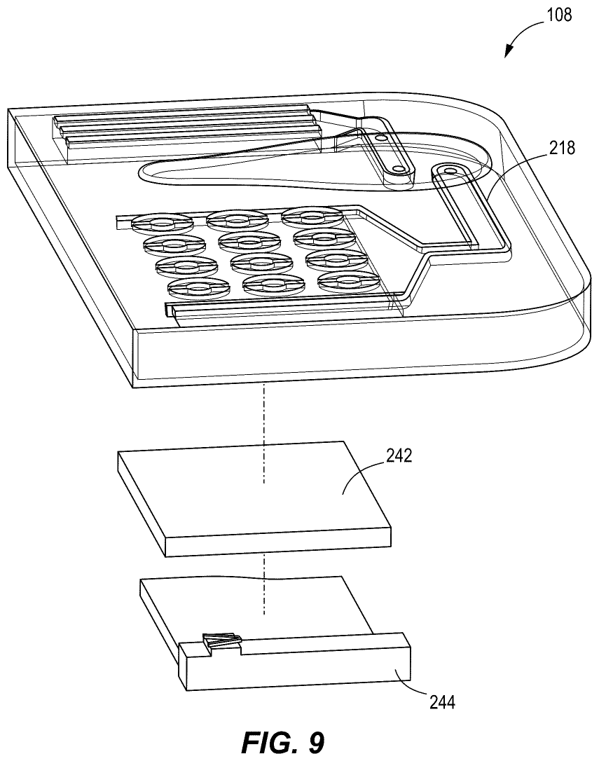

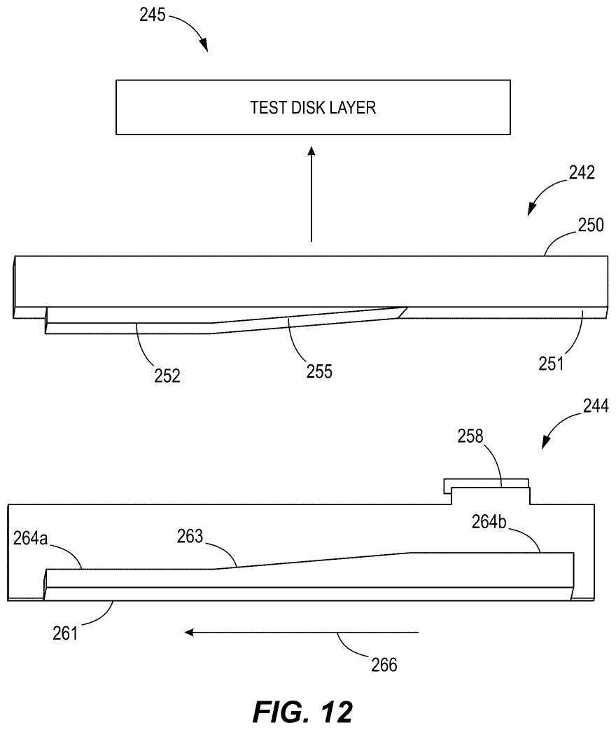

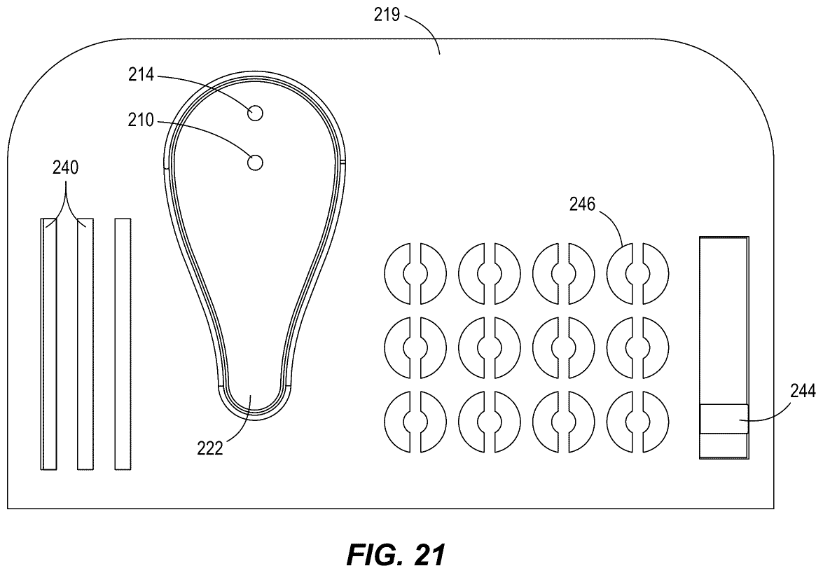

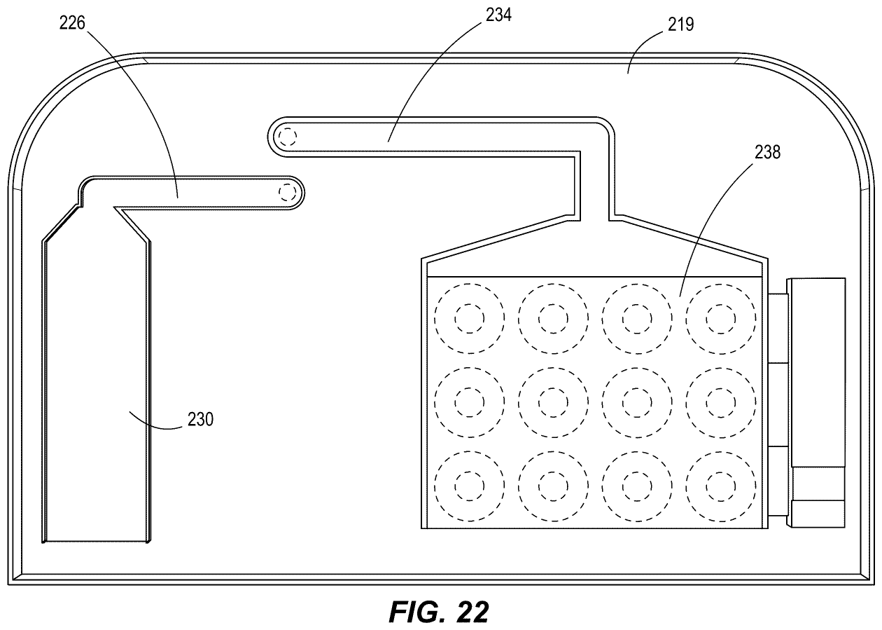

FIGS. 1-12 illustrate a urine collection and analysis assembly 100 according to an embodiment of the present disclosure. The urine collection and analysis assembly 100 includes a specimen cup 104 designed to capture and store urine and an assay base 108 removably couplable to the specimen cup 104 for analyzing the collected urine sample. FIGS. 21-23 illustrate additional views of the assay base 108.

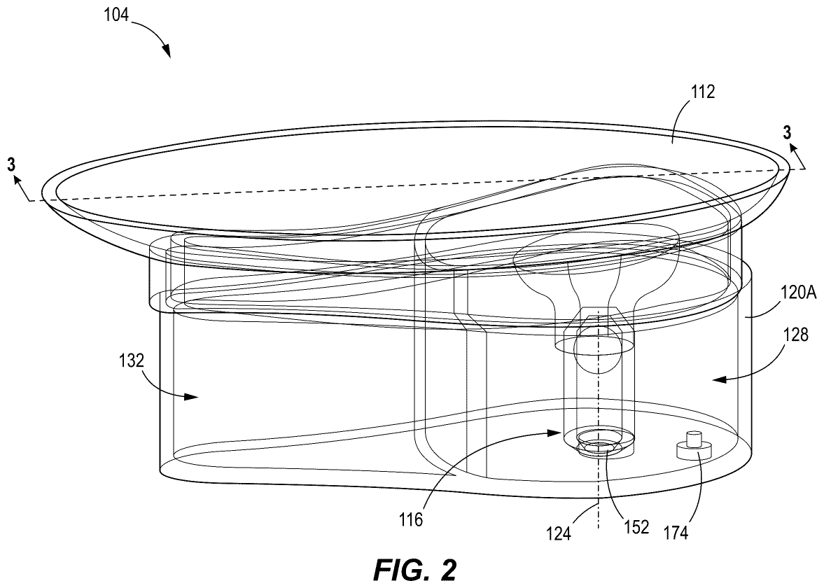

With respect to FIGS. 2-8, the specimen cup 104 includes a saddle 112, an inner housing 116, and an outer housing 120. The saddle 112 is fluidly coupled with both the inner housing 116 and the outer housing 120. The inner housing 116 is supported within the outer housing 120. As discussed in further detail below, each of the saddle 112, inner housing 116, and outer housing 120 may be sealingly coupled together. In other embodiments, the saddle 112 may be removably coupled to the inner and outer housings 116, 120.

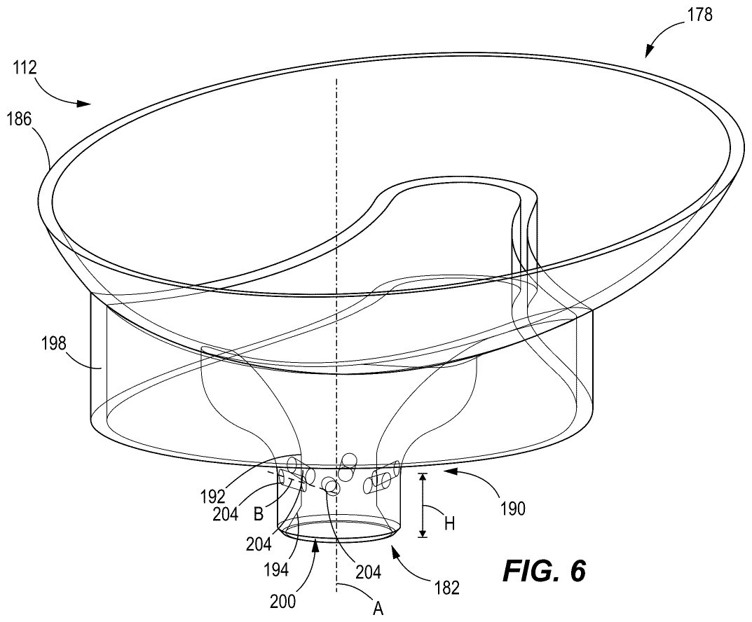

With specific reference to FIG. 2, the inner housing 116 defines a first collection chamber 124 for receiving an initial void (IV) of urine of the user. The first collection chamber 124 may receive between 10 ml and 30 ml of fluid. In one construction the first collection chamber 124 may receive 10 ml to 15 ml of fluid. The outer housing 120 further defines a second collection chamber 128 for receiving a mid-stream-clean-catch (MSCC) of urine and an overflow chamber 132 for receiving excess urine from the second collection chamber 128. The outer housing 120 may include an overflow bore 130 (see FIG. 6) disposed between a wall 170 of the second collection chamber 128 and the overflow chamber 132. The second collection chamber 128 may receive between 15 ml and 100 ml of fluid before overflowing. It will be appreciated that the fixed volumes of the first collection chamber 124 and the second collection chamber 128 allow for predetermined volumes of the initial void and the MSCC urine to be captured and tested. In some embodiments, the overflow chamber 132 may include a liquid absorbing material (e.g., cotton, cloth, a superabsorbent polymer, etc.) to reduce liquid movement within the specimen cup 104. In some embodiments the specimen cup 104 may further include a lid or other sealing means to prevent the spillage of urine during transportation.

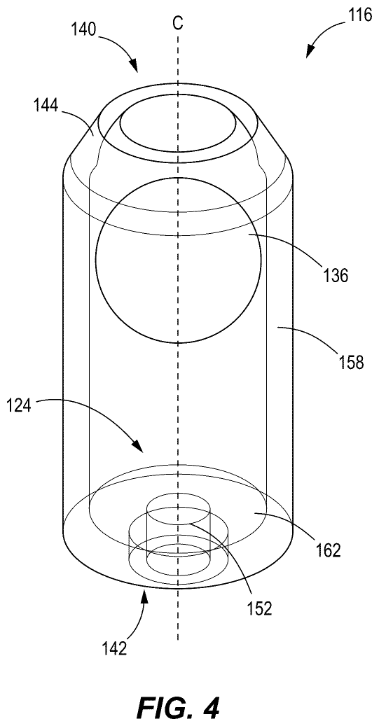

With reference to FIG. 4, the inner housing 116 further includes a first end, a second end opposite the first end, a central axis C extending between the first end and the second end, and a valve element 136 movably disposed within the first collection chamber 124. The first end defines an inlet 140 and the second end defines an outlet 142. The first end has a first, tapered portion 144 extending from the inlet 140 of the first collection chamber 124 towards the second end and a second portion 158 extending from the first portion 144 to the second end. The inner and outer surfaces of the first portion 144 are generally tapered relative to the central axis C. The second portion 158 is a generally cylindrical portion and therefore the inner and outer surfaces thereof have generally uniform dimensions. A first port 152 selectively seals the outlet 142 of the inner housing 116.

The valve element 136 is substantially spherical and formed from a material that is buoyant in urine. Said another way, the valve element 136 may be less dense than urine. The valve element 136 is configured to move from the second end to the first end as urine enters the first collection chamber 124. Thus, the second portion 158 is formed with a diameter greater than the valve element 136. When the valve element 136 reaches the first end, the valve element 136 is configured to close or seal the inlet 140. Therefore, the first portion 144 has a dimension that is equal to or less than the dimension of the valve element 136. During operation, the initial void of urine may flow from the inlet 140 and around the valve element 136. As the first collection chamber 124 is filled, the buoyant valve element 136 may rise along the central axis C within the first collection chamber 124 and eventually engage with the first portion 144 to close the inlet 140. Upon filling the first collection chamber 124, the valve element 136 and the first portion 144 may form a seal, preventing additional urine from flowing into the first collection chamber 124. In the illustrated embodiment, the valve element 136 is a ball valve. In other embodiments other one-way valve shapes and structures may be incorporated.

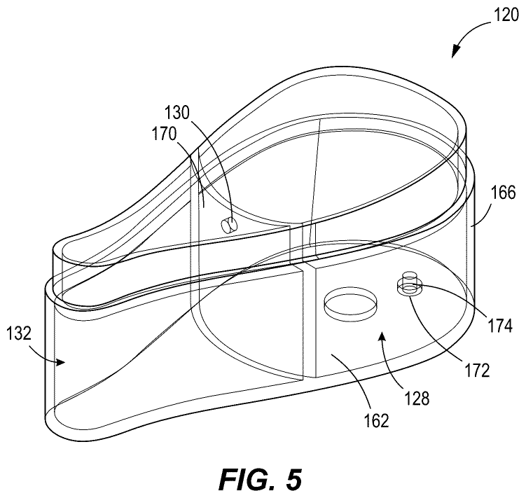

As shown in FIG. 5, the outer housing 120 includes a base wall 162, an outer wall 166 extending from a base wall 162 (e.g., bottom wall), and inner wall 170 extending from the base wall 162. The outer wall 166 defines the periphery of the outer housing 120. The inner wall 170 is a dividing wall extending between separated sides of the outer wall 160. The inner wall 170 separates the second collection chamber 128 from the overflow chamber 132. The second collection chamber 128 and the overflow chamber 132 are fluidly coupled via the overflow bore 130 extending through the inner wall 170. The overflow bore 130 is spaced apart from the base wall 162, such that the second collection chamber 128 is able to fill to the location of the overflow bore 130 before urine is directed to the overflow chamber 132. The base wall 162 includes an aperture 172 extending therethrough and a second port 174. The aperture 172 is positioned within the second collection chamber 128 and is configured to receive the inner housing 116. The base wall 162 is selectively opened via the second port 174.

With reference to FIG. 6, the saddle 112 includes an inlet end 178, an outlet end 182 opposite the inlet end 178, a central axis A extending between the inlet end 178 and the outlet end 182. A first portion 186 extends from the inlet end 178 towards the outlet end 182 and a second portion 190 extends from the first portion 186 towards the outlet end 182. The first portion 186 is a funnel portion with a dimension that gradually decreases in size from the inlet end 178 towards the second portion 190. The second portion 190 has an outer surface and an inner surface. In the illustrated embodiment, the second portion 190 is generally cylindrical, but in other embodiments, the second portion 190 may have other shapes. The outer surface has a generally uniform outer dimension from the first portion 186 towards the outlet end 182. The inner surface has a first inner surface portion 192 extending from the first portion 186 towards the outlet end 182 and a second inner surface portion 194 extending from the first inner surface portion 192 to the outlet end 182. The first inner surface portion 192 has a generally uniform inner dimension, while the second inner surface portion 194 has an inner dimension that gradually increases from the first inner surface portion 192 to the outlet end 182. Accordingly, the second inner surface portion 194 defines a generally tapered surface. The saddle 112 further defines a periphery wall 198 extending between the first portion 186 and the second portion 190. The periphery wall 198 defines a periphery of the saddle 112 and is sized and shaped to engage and seal with the outer wall 166 of the outer housing 120.

In the illustrated embodiment, the second inner surface portion 194 defines a first outlet 200 at the outlet end 182 and the first inner surface portion 192 defines a plurality of second outlets 204 that are spaced apart from the first outlet 200. The second outlets 204 are concentrically distributed about the second portion 190. In the illustrated embodiment, the saddle 112 includes six second outlets 204 arranged circumferential around the central axis A. Additionally, the second outlets 204 are separated from the first outlet 200 by a height H. The second outlets 204 are also angled relative to the central axis A. That is, each of the second outlets has central axis B that forms an obtuse angle relative to the central axis A that faces the first outlet 200. In other words, the second outlets 204 each include a first end at the wall of the second portion 190 and a second end opposite the first end. The second end is positioned higher than the first end. It will be appreciated that the height H of the second outlets 204 and obtuse angle formed between the axes A, B between the first outlet 200 and the second outlets 204 allows for fluid to initially flow through the first outlet 200 without flowing into the second outlets 204.

The saddle 112 is removably coupled to the specimen cup 104. The periphery wall 198 of saddle 112 engages the outer wall 166 of the outer housing 120 to enclose the second and the overflow chambers 128, 132. The second inner surface portion 194 of the saddle 112 engages the tapered portion 144 of the inner housing 116 such that the first outlet 200 of the saddle 112 is fluidly connected to the inlet 140 of the first collection chamber 124. Accordingly, during operation a user may urinate into the funnel portion 186 of the saddle 112, which will direct urine toward the outlets 200, 204. Due to the height and orientation of the outlets 200, 204, respectively, the urine will initially be directed towards the first outlet 200. Said another way, the initial void of urine will be directed towards the first collection chamber 124 and the mid-stream-clean-catch of urine will be directed towards the first collection chamber 124 when the first collection chamber 124 is full and the valve element 136 closes the same. Moreover, the valve element 136 is movable from a first position in which the valve element 136 is positioned adjacent to the second end of the inner housing 116 and a second position in which the valve element 136 is positioned adjacent to the first end of the inner housing 116 and engages the first outlet 200. The valve element 136 is positioned in first position when the first collection chamber 124 is empty and is in the second position to close the first collection chamber 124. As the initial void fills the first collection chamber 124, the valve element 136 moves from the first position to the second position. The mid-stream-clean-catch flows to the second collection chamber 128 through the second outlets 204 when the valve element 136 is in the second position. To prevent leakage of urine between the saddle 112 and the inner housing 116, the interface between the saddle 112 and the inner housing 116 may be sealed using a gasket or another sealing means such as an adhesive. Similarly, to prevent leakage of urine between the saddle 112 and outer inner housing 120, the interface between the saddle 112 and the outer housing 120 may be sealed using a gasket or another sealing means such as an adhesive.

During operation, the specimen cup 104 is operable to collect and automatically isolate and store urine for a user. For brevity's sake, the initial void of urine stored within the first collection chamber 124 will herein be referred to as “the initial void” or “the IV.” Similarly, the urine stored within the second collection chamber 128 will herein be referred to as “the mid-stream-clean-catch” or “the MSCC.” The initial void enters the saddle 112 and is directed to the first collection chamber 124 via the first outlet 200. Once the first collection chamber 124 is filled, the flow of liquid is restricted from the first outlet 200 by the valve element 136, which has closed the inlet 140. The liquid flow is then redirected to the plurality of second outlets 204 and into the second collection chamber 128. Once the second collection chamber 128 is filled, fluid will flow out of the overflow bore 130 into the overflow chamber 132. It will be appreciated that the first collection chamber 124 is fluidly separated from the second collection chamber 128. Accordingly, the IV isolated within the first collection chamber 124 does not contaminate the mid-stream-clean-catch directed to the second collection chamber 128.

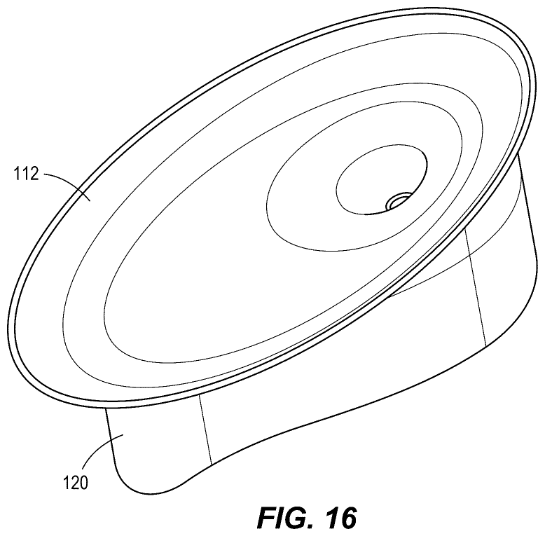

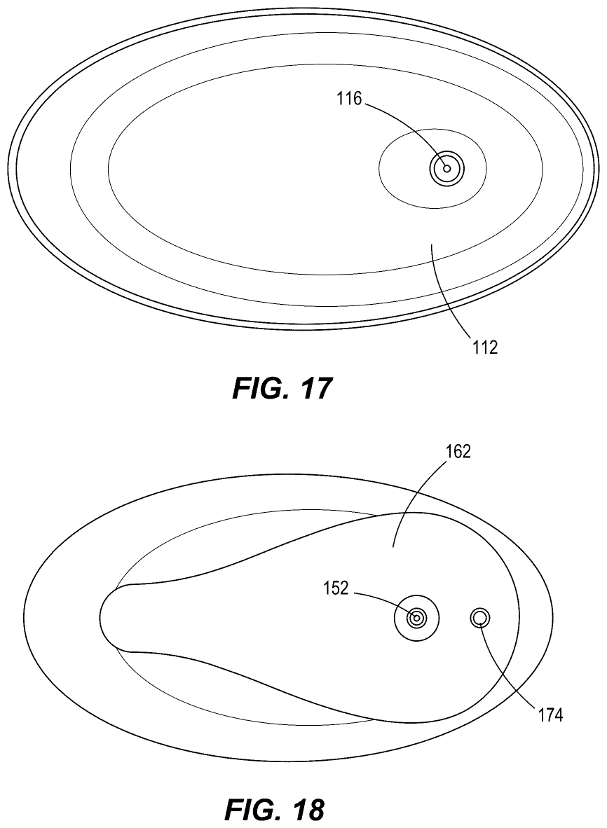

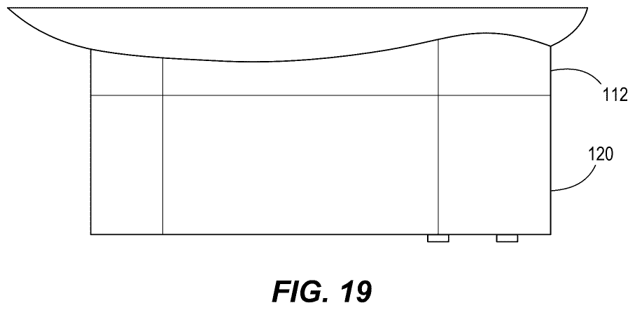

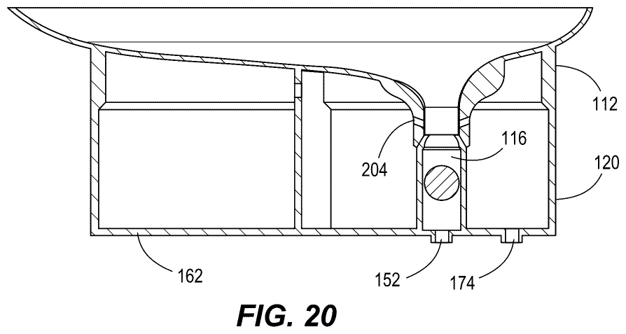

FIGS. 16-20 illustrate another embodiment of the specimen cup for use with assay base 108. The specimen cup of FIGS. 16-20 is the same as the specimen cup of FIGS. 1-13 except that the inner housing 116 is integrally formed with the outer housing 120.

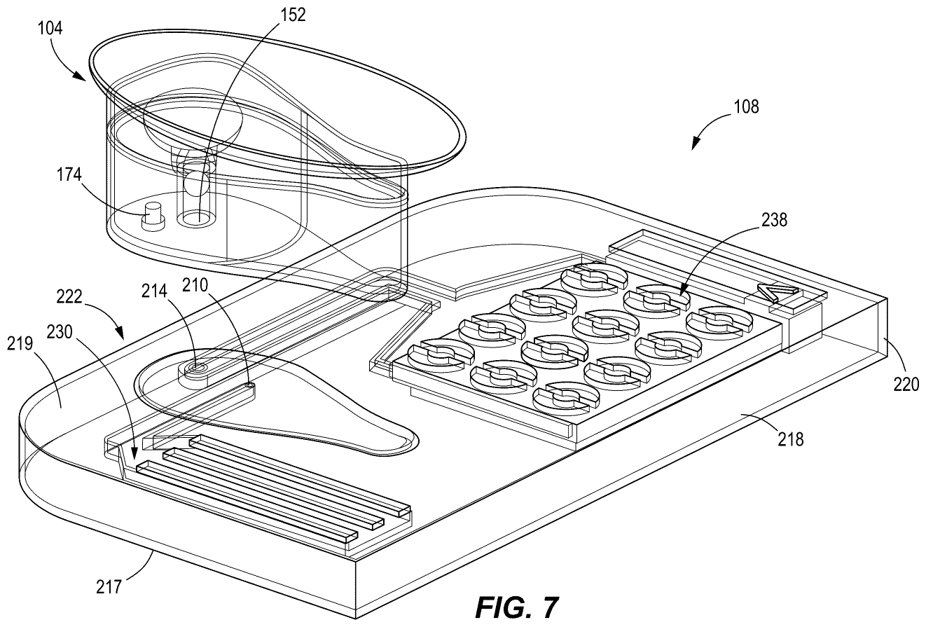

With reference to FIGS. 7-12, the specimen cup 104 is configured to engage an assay base 108. The assay base 108 is configured to analyze both the initial void and the mid-stream-clean catch stored within the specimen cup 104, as will be discussed in greater detail below.

With continued reference to FIGS. 7-12, the assay base 108 includes a base housing 218 including a bottom wall 217 (e.g., a base wall), a top wall 219, and an outer wall 220 between the bottom wall 217 and the top wall 219. The base housing 218 encloses an interior thereof. A recess 222 is in the top wall 219, and apertures extends through the top wall 219. The apertures are positioned within the recess 222 of the top wall 219. A first of the apertures supports a first port actuator 210 and a second of the apertures supports a second port actuator 214. The base housing 218 defines a first channel 226, a first testing area 230, a second channel 234, and a second testing area 238. The first channel 226 is in communication with both the first aperture, via the first port actuator 210, and the first testing area 230. The second channel 234 is in communication with both the second aperture, via the second port actuator 214, and the second testing area 238 In some embodiments, the recess 222 and/or portions of the top wall 219 may include locking tabs configured to secure the specimen cup 104 to the assay base 108 to prevent accidental spillage and premature detachment of the specimen cup.

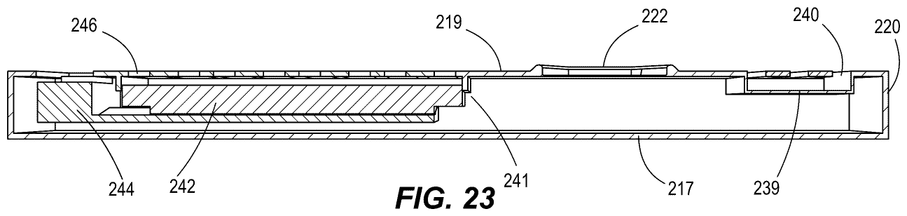

In the illustrated embodiment, the first channel 226 and the first testing area 230 are defined in the top wall 219, as shown in FIG. 23. That is, the first channel 226 is positioned within the top wall 219. Similarly, the first testing area 230 includes a plurality of recesses 239 positioned within the top wall 219 and in communication with the first channel 226. In other embodiments, the first channel 226 and the first testing area 230 may be positioned within the interior of the base housing 218 and defined by structure that is beneath the top wall 219 (e.g., between the top wall 219 and the bottom wall 217). In the illustrated embodiment, the top wall 219 includes openings or viewing ports 240 that are positioned over the recess 239. In some embodiments, all or a portion of a top surface of the top wall 219 may be transparent to enable the first channel 226 and the first testing area 230 to be viewed therethrough. In other embodiments, all or a portion of a top surface of the top wall 219 may be transparent to enable the first channel 226 and the first testing area 230 to be viewed therethrough.

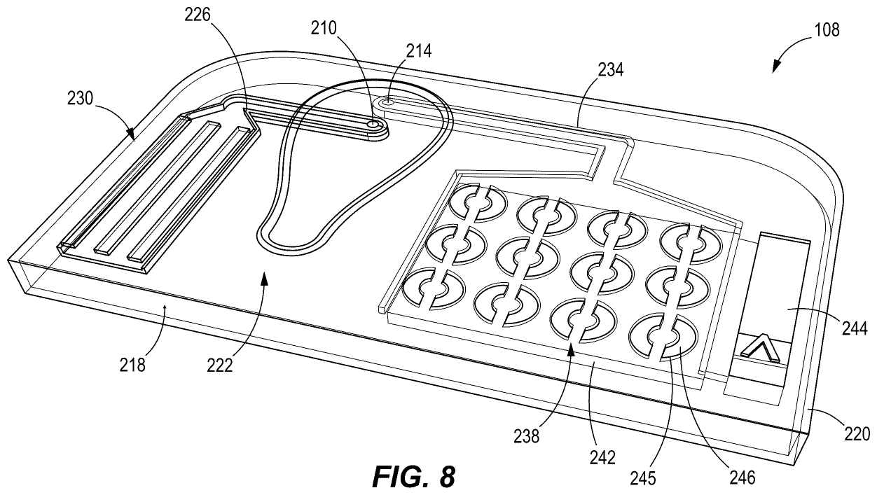

As shown in FIG. 8, the first channel 226 is an initial void channel that guides the initial void from the first collection chamber 124 to the first testing area 230, where any substrate diffusion-based urine testing can occur. For example, the first testing area 230 may include one or more substrates configured for any urinalysis test that can be performed using urine test strips, urine dipsticks, Enzyme-Linked Immunosorbent Assay (ELISA), lateral flow, PCR, and/or NAATs. The one or more substrates may be positioned within the recess 239. Once the initial void sample has completed its exposure to the various testing substrates, it is directed from the base housing 218 into urine-absorbing material (not shown) within the interior of the base housing 218 to provide for greater hygienic handling and ease of disposal. Due to the openings 240 in the top wall 219, the substrates are viewable therethrough for analysis. In illustrated embodiments, the first channel 226 or the first testing area 230 may include a viewing port operable for visual, microscopic, and/or camera assisted inspection. The viewing port may include a light operable in a plurality of wavelengths such that a user may illuminate the initial void. In some embodiments, the viewing port may include a lens shaped to magnify the image provided by the viewing port such that a user may visually inspect the sample. In other embodiments, the viewing port may be couplable with a microscope or camera (such as a phone camera) for use in visual inspection of the initial void sample. The visual inspection operation may include, at least, a detection of particulate, a color test, and a characterization of sample clarity. For example, the visual inspection operation may be used to detect the presence of blood or microplastics within the provided initial void sample.

In the illustrated embodiment, the second channel 234 and the second testing area 238 are defined in the top wall 219, as shown in FIG. 23. That is, the second channel 234 is positioned within the top wall 219. Similarly, the second testing area 238 includes a recess 241 positioned within the top wall 219 and in communication with the second channel 234. In other embodiments, the second channel 234 and the second testing area 238 may be positioned within the interior of the base housing 218 and defined by structure that is beneath the top wall 219 (e.g., between the top wall 219 and the bottom wall 217).

The second channel 234 is a mid-stream-clean-catch channel that directs the mid-stream-clean-catch channel fluid from the second collection chamber 128 to the second testing area 238. The second testing area 238 includes a plurality of testing members 245 stored within hemispherical openings 246. In the illustrated embodiment, the hemispherical openings 246 are in the top wall 219, as shown in FIG. 23. The testing members 245 are selectively exposed to a growth medium contained on a platform 242. The platform 242 is movable relative to the testing members 245 via a lifting actuator 244. The platform 242 and the lifting actuator 244 are positioned within the recess 241 of the top wall 216. As shown, a height of the recess 241 of the second testing area 238 is greater than a height of the recess 239 of the first testing area 230.

The platform 242 includes a growth medium. The growth medium is configured to retain at least a portion of the MSCC sufficient to expose the growth medium to the urine sample for the purposes of inoculation. The growth medium may be a general-purpose media such as nutrient agar, LB agar, or tryptic soy agar (TSA). In some constructions, the growth medium may additionally or alternatively be an enriched media, a selective Media, a differential media, a minimal Media, a Specialized Media, or a Liquid Media. The growth medium may be divided into discrete sections to support multiple types of media.

The testing members 245 may be formed of cotton, paper, or similar media. The testing members 245 may include a control testing member and subsets of testing members with various antibiotics and testing compounds. For example, one or more of the testing members may also include (e.g., are dusted/coated/impregnated with) antibiotics, and one or more of the testing members 245 may also include testing compounds (e.g., compounds associated with NAAT/PCR-based testing or microplastic particulate detection) in addition to or in lieu of antibiotics. Accordingly, the testing members 245 are configured to inoculate and/or detect organisms in the MSCC in a growth medium. In the illustrated embodiment, the top surface of the top wall adjacent the second testing area 238 may include a graphic overlay to easily allow a user to determine the size of the “zone of exclusion” created by the antibiotic testing members when they inhibit the growth of the organism(s).

The lifting actuator 244 is configured to move the platform 242 between a first position and a second position. In the first position, the platform 242 is spaced apart from the plurality of testing members 245 such that the growth medium is also spaced apart from the plurality of testing members 245. In the second deposition, the platform 242 is positioned adjacent to or otherwise contacts the testing members 245 such that the growth medium contacts the testing members 245 and thereby exposes the MSCC to the contents of the testing members 245.

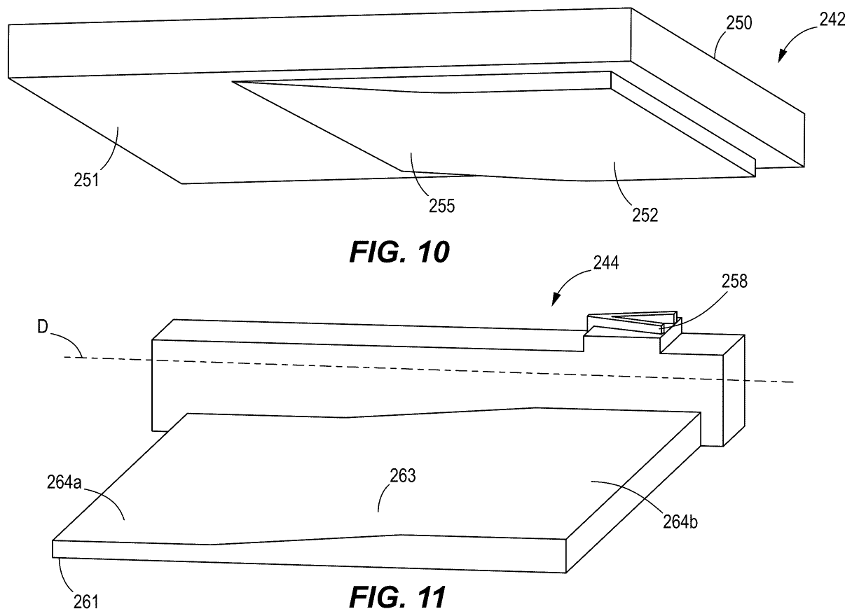

With specific reference to FIGS. 10-12, the platform 242 includes a body with a generally upper surface 250, a first lower surface 251, a second lower surface 252, and a ramp or angled surface 254 coupled to and extending between the first lower surface 251 and the second lower surface 252. The angled surface 255 is positioned at angle relative to a plane defined by the upper surface 250. In the illustrated embodiment, the upper surface 250, the first lower surface 251 and the second lower surface 252 are each planar or flat. In other embodiments, these surfaces may have other configurations.

As illustrated in FIG. 11, the lifting actuator 244 includes a body having a generally lower surface 261, a first upper surface 264a, a second upper surface 264b, and a ramp or an angled surface 263 extending between the first and second upper surfaces 264a, 264b. The angled surface 263 is positioned at an angle relative a plane defined by the lower surface 261. A handle portion 258 (e.g., button portion, knob portion, etc.) is coupled to the body. The handle portion 258 defines a movement axis D.

The platform 242 is movably supported on the actuator 244. Specifically, in the first position, the platform 242 is supported on the actuator 244 such that the second lower surface 252 is supported by the first upper surface 264a, the first lower surface 251 is supported by the second upper surface 264b, and the angled surfaces 254, 263 are engaged with (or otherwise positioned relative to) one another. In the second position, the platform 242 is supported on the actuator 244 such that the second lower surface 252 is supported by the second upper surface 264b and the first lower surface 251 and the angled surface 254 are generally spaced apart from the actuator 244. The user moves the platform 242 from the first position to the second position by moving (e.g., pushing) the handle portion 258 of the lifting actuator 244 in the direction of arrow 266 (e.g., toward the second channel 234). Accordingly, the movement of the lifting actuator 244 causes the corresponding angled surface 255, 262 to slide over one another and raise the platform 242 by a predetermined height.

In use, the specimen cup 104 is coupled to the base assay 108. That is, the specimen cup is positioned within the recess 222. Accordingly, the first port 152 of the first collection chamber 124 engages the first port actuator 210 to enable fluid communication between the first collection chamber 124 and the first channel 226. Also, the second port 174 engages the second port actuator 210 to enable fluid communication between the second collection chamber 128 and the second channel 234. Thus, the initial void of the first collection chamber 124 is guided from the first collection chamber 124 to the first testing area 230 via the first channel 226, and the mid-stream-clean-catch of the second collection chamber 128 is guided from the second collection chamber 128 to the second testing area 238 via the second channel 234.

The first port 152 and the second port 174 may each be formed using a semi-permeable seal or one-way valve bodies. The semi-permeable seals may be operable to release the fluid within the respective first collection chamber 124 and second collection chamber 128 when pierced or actuated upon. In other embodiments, the first port 152 and/or the second port 174 may be formed using another openable seal or one-way valve bodies known in the art. The first port 152 and the second port 174 are thus simultaneously openable via the first port actuator 210 and the second port actuator 214, respectively, such that the initial void of urine and the mid-stream-clean-catch of urine move to the first testing area 230 and the second testing area 238, respectively, via gravity.

As noted above, the initial void is automatically analyzed at the first testing area. That is, the first channel 226 guides the initial void to the first testing area for automatic analysis.

In contrast, the mid-stream-clean-catch is analyzed once the growth medium is moved to the second position. That is, initially, the platform 242 is in the first position such that the grown medium is spaced apart from the testing members 245. As the mid-stream clean catch is guided to the second testing area 238, the urine is thus guided to the growth medium while the platform 242 is in the first position. After the mid-stream-clean-catch has been guided to the second testing area 238, the lifting actuator 244 is moved along the axis D to move the platform 242, and therefore the growth medium, from the first position to the second position. As the platform 242 moves from the first position to the second position, excess urine drains the MSCC sample urine into a storage cavity defined by and disposed within the base housing 218. Accordingly, the growth medium is selectively exposed to the testing members 245 for analysis. The testing members 245 may be kept separate from the growth medium during the flooding phase in order to keep their contents from spilling into adjacent test areas.

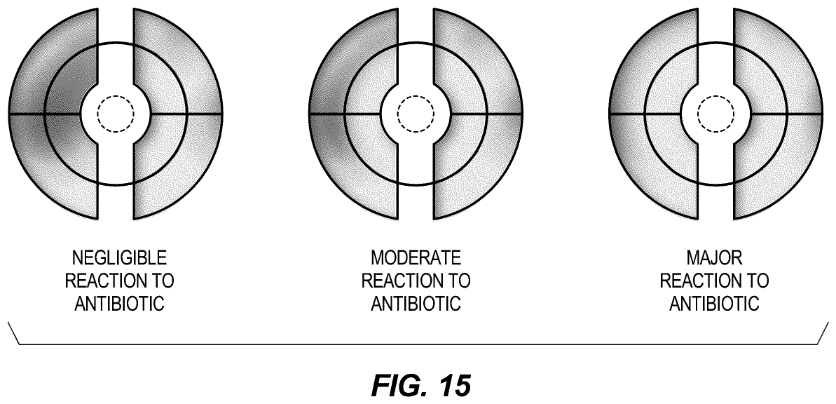

To evaluate the effect of a specific compound on an individual testing member 245, and thereby the organisms within the MSCC sample, a user must evaluate the inoculation or growth of an area of the growth medium contacted by an individual testing member 245. A user may be directed to identify a “zone of exclusion” on the platform 242. As illustrated in FIG. 15, each “zone of exclusion” is defined by an area of no growth or of limited growth after a period of time. For example, in one exemplary “zone of exclusion” using a fully effective antibiotic test disk, bacteria did not grow in the zone of exclusion. The type of antibiotic corresponding to the first area is known, and accordingly the effectiveness of that specific antibiotic can be qualified. In another example, in a second area, the size of the “zone of exclusion” is reduced compared to the first area. Accordingly, the bacteria grew within the sampled area at a reduced rate. It may be determined that the corresponding antibiotic or other compound was partially effective. In a third example using a control, the area of the platform 242 has fully grown bacteria and no “zone of exclusion” can be identified.

As illustrated in Table 1 below, several organisms commonly found in UTIs grow within temperature ranges between 50- and 113-degrees Fahrenheit. Accordingly in some embodiments the lifting actuator 244, or a portion of the base housing 218, may further include a heating element configured to maintain the temperature of the assay base 108 within the growth temperature ranges listed below. The heating element may be attached to an electronic controller, a temperature sensor, and a power source.

TABLE 1

| | Growth | |
|---|---|---|
| | Temp | |
| Organism | Range (° F.) | Common Role in UTIs |
| Escherichia coli | 50-113 | Most common cause of UTIs, |
| | | particularly cystitis. |
| Klebsiella | 50-104 | Frequently associated with complicated |
| pneumoniae | | UTIs. |
| Proteus mirabilis | 68-113 | Known for causing UTIs in patients |
| | | with catheters. |
| Staphylococcus | 50-104 | Commonly causes UTIs in sexually |
| saprophyticus | | active young women. |
| Enterococcus | 50-113 | Associated with complicated UTIs and |
| faecalis | | hospital settings. |
| Pseudomonas | 68-107.6 | Causes UTIs in immunocompromised |
| aeruginosa | | patients & catheterized individuals. |
| Candida albicans | 68-104 | Causes fungal UTIs, typically in |
| | | immunocompromised individuals. |

In the illustrated embodiment, the platform 242, growth medium, and actuator 244 are preinstalled within the assay base 108. In some embodiments a separate media cartridge including the growth medium may be installed into the assay base 108 at the time of use.

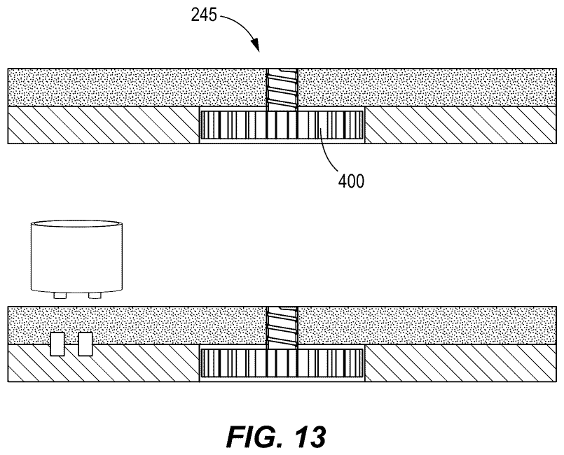

The lifting actuator for raising the platform 242 may have other configurations. FIG. 13 illustrates another exemplary embodiment of the lifting actuator 244 of the second testing area 238. The embodiment includes a gear 400 rotatable by a user to raise the platform 242 using a screw 404. In other embodiments, other actuation mechanisms may be implemented to raise the platform 242 into the testing members 245 such as a pneumatic piston, a spring, a lever, etc. In still other embodiments, the testing members 245 may be lowered onto the platform 242 instead of raising the growth medium. In yet other embodiments, locking tabs may be connected to the recess 222 to prevent relative movement of the platform 242 and the testing members 245 when the specimen cup 104 is not coupled to the assay base 108.

The specimen cup 104 may have other configurations including different shapes and sizes operable for different users. Particularly, the shape of the saddle 112, and the dimensions of the inner housing 116 and the outer housing 120 may be altered depending on the gender and/or species of the user. For example, a specimen cup modified for use by an animal (e.g., horse, dog, cat, etc.) may be modified to decrease the height of the specimen cup to allow for easier accessibility for the animal. Similarly, the surface area of the saddle 112 may be increased or decreased depending on the application of the collection assembly 100. In some embodiments, the volume defined by the inner housing 116 and the outer housing 120 may also accordingly be adjusted to the expected volumetric output of the user. Additionally, in some embodiments, the assay base 108 may also be modified to correspond with the size or shape of the specimen cup 104.

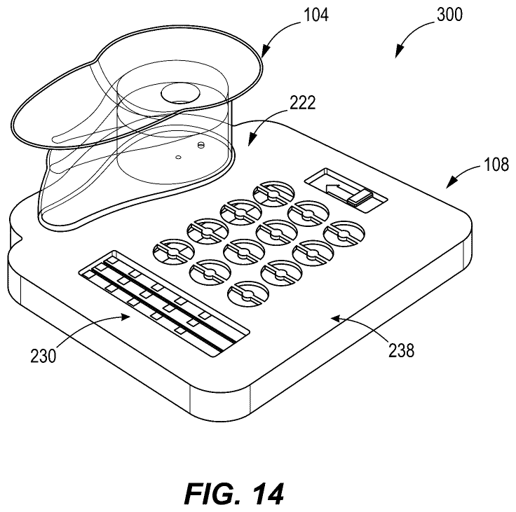

FIG. 14 illustrates another embodiment of the urine collection and analysis assembly 300. The assembly 300 similar to the urine collection and analysis assembly 100, with like parts having the same reference numerals. It should be noted that the configuration and orientation of the recess 222, the first testing area 230, and the second testing area 238 may be arranged differently. In some embodiments the assay base 108 may only include one testing area.

The urine collection and analysis assembly 100 may be manufactured using a type of medical grade plastic. For example, the elements of the urine collection assembly may be formed from one or more of the following materials: polypropylene (PP), by polyethylene terephthalate (PET, PETE), high density polyethylene (HDPE), low density polyethylene (LDPE) and/or polyvinyl chloride (PVC, Vinyl). At least some of these materials are appropriate for various forms of sterilization as urine collection assembly may be sterilized at some point prior to use by the patient. The list of materials above is not exhaustive, and therefore elements of the urine collection assembly may be formed from any suitable material.

The urine collection and analysis assembly 100 may be manufactured by assembling multiple parts, which may be formed by blow molding, injection molding, extrusion blow molding, injection blow molding, injection stretch blow molding, die forming, molding, thermoforming, or vacuum forming. Other forms of manufacturing may include: 3D printing, extrusion, and UV 3D resin printing/manufacturing. The manufactured parts may be assembled into the proper configuration for use using, for example, sonic welding or any other suitable coupling method.

Sterilization may occur during manufacturing or at the pre-packaging stage. Sterilization may occur by chemical sterilization (e.g., with one or more of nitrogen dioxide (NO2), ethylene oxide (EO), utilizing hydrogen peroxide, and peracetic acid). Alternatively, sterilization may also be accomplished by radiation sterilization (e.g., gamma radiation, electron beam processing, high-energy X-rays, or other forms of irradiation), autoclaving, steam sterilization, dry heat sterilization, or any other FDA approved sterilization method.

The parts of the urine collection and analysis assembly 100 that will likely be in contact with the urine specimen may be coated with a hydrophobic coating or compound. The hydrophobic coating may help to ensure that a majority of the initial void will pass through the funnel portion 186 of the saddle 112 and into the first collection chamber 124. In this way, the first collection chamber 124 will capture the cellular debris and other biological material that is important to properly performing a NAAT test on the initial void but that would otherwise be viewed as a contaminant to the MSCC portion of the sample. In other or additional embodiments, the materials of the urine collection assembly may be sufficiently hydrophobic on their own and not require a separate application of a hydrophobic coating.

The benefits of the urine collection container include its capability of automatically collecting and separating a “initial void” and a “mid-stream clean-catch” sample from a patient, minimizing the cognitive load and physical requirements placed on the patient thereby reducing the chances for error or contamination, and improving patient compliance and patient satisfaction. Moreover, the urine collection assembly enables improvements in the collection process to provide better or more high-quality samples for testing. Providing better samples and separate samples (e.g., samples from the initial void and the mid-stream clean-catch) for testing will provide for improved results in the form of more accurate diagnosis and prescribed course of treatment.

It will be further appreciated that the urine analysis capability of the assay base 108 reduces the logistical steps necessary for testing collected urine samples.

Various features are set forth in the following claims.

What is claimed is:

1. A urine collection and analysis assembly comprising:
a specimen cup having a first collection chamber and a second collection chamber; and
an assay base couplable with the specimen cup, the assay base including a first testing area including one or more testing substrates and a second testing area having one or more testing members,
wherein coupling the specimen cup and the assay base fluidly connects the first testing area to the first collection chamber such that an initial void of urine runs over the one or more testing substrates for analyzing the initial void of urine and the second testing area to the second collection chamber such that a mid-stream-clean-catch of urine is exposed to the one or more testing members for analyzing the mid-stream-clean-catch.

2. The urine collection and analysis assembly of claim 1, wherein the specimen cup further includes a valve element configured to separate the initial void of urine from the mid-stream-clean-catch of urine.

3. The urine collection and analysis assembly of claim 2, wherein the valve element is positioned with and movable relative to the first collection chamber.

4. The urine collection and analysis assembly of claim 1, wherein the specimen cup further includes a saddle having a first outlet couplable to the first collection chamber and a second outlet coupled to the second collection chamber.

5. The urine collection and analysis assembly of claim 1, wherein the second testing area includes an actuator and a growth medium, the growth medium is configured to receive at least a portion of the mid-stream-clean-catch, and the actuator configured to move the growth medium from a first position in which the growth medium is spaced apart from the one or more testing members and a second position in which the growth medium engages the one or more testing members.

6. The urine collection and analysis assembly of claim 1, wherein the first collection chamber includes a first port that is selectively opened via a first port actuator of the assay base and the second collection chamber includes a second port that is selectively opened via a second port actuator of the assay base.

7. The urine collection and analysis assembly of claim 6, wherein the first port and the second port are simultaneously openable via the first port actuator and the second port actuator, respectively, such that the initial void of urine and the mid-stream-clean-catch of urine move to the first testing area and the second testing area, respectively, via gravity.

8. The urine collection and analysis assembly of claim 6, wherein the first port actuator is in fluid communication with the first testing area via a first channel positioned within the assay base and the second port actuator is in fluid communication with the second testing area via a second channel positioned within the assay base.

9. A specimen cup configured to receive a urine sample, the specimen cup comprising:
an outer housing including
a base wall;
an outer wall coupled to and extending from the base wall, the outer wall defining a perimeter of the specimen cup;
an inner housing positioned within the outer housing and including
a first end;
a second end opposite the first end and supported by the base wall of the outer housing;
a valve element movably positioned with the inner housing;
a first collection chamber defined by the inner housing and configured to receive an initial void of the urine sample;
a second collection chamber defined within the outer housing between the inner housing and the outer wall, the second collection chamber in fluid communication with the first collection chamber and configured to receive a mid-stream-clean-catch of the urine sample;
a first port positioned at the second end of the inner housing accessible through the base wall and configured to selectively open the first collection chamber; and
a second port accessible through the base wall and configured to selectively open the second collection chamber.

10. The specimen cup of claim 9, wherein the first port and the second port are simultaneously openable, such that the initial void and the mid-stream-clean-catch exit the specimen cup via gravity.

11. The specimen cup of claim 9, further comprising a saddle configured to guide the urine sample to the specimen cup and having a first outlet couplable to the first collection chamber and a second outlet coupled to the second collection chamber.

12. The specimen cup of claim 11, wherein the valve element is movable from a first position in which the valve element is positioned adjacent to the second end and a second position in which the valve element is positioned adjacent to the first end and engages the first outlet, the valve element being positioned in first position when the first collection chamber is empty and being in the second position to close the first collection chamber, and wherein as the initial void fills the first collection chamber, the valve element moves from the first position to the second position.

13. The specimen cup of claim 12, wherein the mid-stream-clean-catch flows to the second collection chamber through the second outlet when the valve element is in the second position.

14. The specimen cup of claim 9, further comprising an overflow chamber in fluid communication with the second collection chamber.

15. The specimen cup of claim 14, wherein an inner wall is coupled to and extends from the base wall, wherein the inner wall extends between opposite sides of the outer wall and separates the overflow chamber from the second collection chamber, and wherein the inner wall includes a bore allowing urine to flow from the second collection chamber to the overflow chamber, the bore in the inner wall being spaced apart from the base wall.

16. An assay base couplable with a specimen cup configured to receive a urine sample, the assay base comprising:
a housing having a base wall and a top wall;
a first port actuator accessible through the top wall;
a first testing area including one or more testing substrates;
a first channel within the housing and in fluid communication between the first port actuator and the first testing area;
a second port actuator accessible through the top wall;
a second testing area having one or more testing members; and
a second channel within the housing and in fluid communication between the second port actuator and the second testing area,
wherein the first testing area is configured to receive an initial void of the urine sample, which runs over the one or more testing substrates for analyzing the initial void, and
wherein the second testing area is configured to receive a mid-stream-clean-catch of the urine sample, which is selectively exposed to the one or more testing members for analyzing the mid-stream-clean-catch.

17. The assay base of claim 16, wherein the top wall includes a recess configured to receive a perimeter of the specimen cup, and wherein the first port actuator and the second port actuator are positioned are positioned within the recess.

18. The assay base of claim 16, further comprising an actuator and a growth medium positioned within the second testing area, the growth medium is configured to receive at least a portion of the mid-stream-clean-catch, and the actuator configured to move the growth medium from a first position in which the growth medium is spaced apart from the one or more testing members and a second position in which the growth medium engages the one or more testing members.

19. The assay base of claim 18, wherein
the actuator includes a body with an angled surface;
the growth medium is positioned on a platform having an angled surface,
the actuator is movable between a first position in which the angled surface of the body is adjacent to the angled surface of the platform and a second position in which the angled surface of the body is spaced apart from the angled surface of the platform, and
movement of the actuator from the second first position to the second position causes the angled surface of the platform to slide over the angled surface of the body to move the growth medium from the first position to the second position and thereby raise the platform by a predetermined height.

20. The assay base of claim 18, wherein the one or more testing substrates of the first testing area automatically analyze the initial void via contact with the initial void.
